(19) United States
(12) Patent Application Publication
(10) Pub. No.: US 2002/0123669 A1
(43) Pub. Date: Sep. 5, 2002

(54) Capacitive pressure sensor

(76) Inventor: Timothy K. Wickstrom, Elk Grove, IL (US)

Correspondence Address: Thomas K. Stine, Esq., WALLENSTEIN & WAGNER, LTD., 311 South Wacker Drive, 53rd Floor, Chicago, IL, 60606-6622, US

(21) Appl. No.: 09/797,388

(22) Filed: Mar. 1, 2001

(51) Int. Cl. A61M 11/00

(52) U.S. Cl. 600/235; 128/200.14

(57) ABSTRACT

A method and apparatus for sensing a pressure difference between a first and second region is disclosed. The apparatus includes a motor assembly having a backplate and a diaphragm, the motor assembly being operably attached between the first and second regions. The motor assembly also having a capacitance responsive to a difference in atmospheric pressure between the first and second regions. A measurement device utilizes the capacitance for detecting the pressure difference between the two regions. The apparatus is adaptable for cooperation with a chemical dispenser for dispensing a prescribed dosage to an individual.

### TECHNICAL FIELD

The present invention relates generally to fluid flowmeters, and, more particularly, to a motor for sensing pressure differential. The present invention further relates to using a motor for measuring a fluid flow volume, such as for dispensing a measured amount of a chemical to an individual.

### BACKGROUND OF THE INVENTION

Flowmeters are instruments for measuring the speeds of fluids. An anemometer is a flowmeter designed to measure the flow of gases. A full bore vortex meter is capable of measuring either liquids or gases.

Flowmeters are used to measure the volume of flow of fluids in a confined space such as a pipe. However, the volume of fluid flow can also be measured in an open channel. Typically, a volume of fluid flowing past a point is measured during a predetermined time. Fluid flow measurement is used in oil and gas pipelines, auto fuel lines for computing miles per gallon usage, and on sea beds to measure the water current.

A common method of fluid flow measurement is the differential pressure (ΔP) meter. There are a variety of industrial differential pressure meters available, e.g., venturi tubes, Dall tubes, orifice plates, etc. These types of differential pressure meters operate on the principle that fluid accelerated through a restriction has its kinetic energy momentarily increased at the expense of its pressure energy. The energy increase or decrease can then be measured.

A venturi meter incorporates a pipe with a smooth and gradual reduction in its cross-sectional area and a similar expansion back to its normal pipe size further downstream. FIG. 1A. The smooth expansion reduces turbulence resulting from eddy currents, thereby diminishing the amount of pressure drop and corresponding loss that may occur. Although the venturi tube is an accurate instrument for measuring pressure difference, it is expensive to manufacture.

A flow nozzle meter has a nozzle-shaped restriction fitted in a pipe that has a smooth, gradual change in the pipe area. FIG. 1B. The nozzle ends abruptly and returns to its full size. A pressure measurement is taken before and after the nozzle.

An orifice plate is relatively simple and less expensive than the flow nozzle or venturi tube. FIG. 1C. It includes a thin plate with a hole near its middle and is mounted across the pipe, perpendicular to the flow direction. Similar to the flow nozzle, pressure measurements are taken before and after the plate.

A variable area flowmeter called a rotameter, utilizes the same principle as the differential pressure meter, i.e., kinetic energy vs. pressure energy. However, in contrast to the fixed restriction size of the differential pressure meter wherein differential pressure changes relate to the flow volume, the rotameter changes the restriction area as the flow volume changes, thus the pressure difference remains constant.

A positive displacement flowmeter operates by trapping a known volume of fluid and counting the number of fluid “packets” that pass as the fluid passes from inlet to outlet. A geared output shaft is connected to a counter for display.

An electromagnetic flowmeter operates on the principle that the electromagnetic field induced in a conductor moving through a magnetic field is proportional to the velocity of the conductor. The electromagnetic flowmeter comprises a non-magnetic stainless steel pipe lined with an insulating material. A magnetic field is produced across the tube by exciting coils arranged around the outside. Liquid passing through the flowmeter becomes the conductor and induces an electromagnetic field. The induced emf is proportional to the fluid velocity.

A turbine flowmeter utilizes a rotor blade operably mounted within the fluid flow wherein the axis of blade rotation is perpendicular to the direction of fluid flow. The angular velocity of the rotating rotor is proportional to the volume of fluid flow. Turbine meters are highly accurate and commonly used for transfer measurement of crude oil and petroleum.

Anemometers measure fluid velocity, more specifically, air or gas velocity. Various types of anemometers can be utilized depending upon the velocity range and accuracy desired. Some of the most common anemometers include: the cup, the propeller and the pitot-static pressure tube.

The cup anemometer is a simple and inexpensive flowmeter consisting of a number of conical or hemispherical cups mounted at the ends of horizontal spokes which radiate from a vertical rotating shaft. The concave surfaces of the cups are influenced by the gas flow, thus causing the shaft to rotate. The shaft is operably connected to a counter device that monitors the number of revolutions of the shaft during a predetermined period, thus calculating the speed of the flowing gas. Alternative display techniques exist for translating the shaft rotations to a voltage for measurement by a voltmeter.

The propeller anemometer is suitable for measuring air flow speeds in the range of 1 to 25 miles per hour. Similar to cup anemometers, the blades of the propeller are attached to a horizontal rotating shaft and directed into the wind by a weathervane-like tail fin. The rotation speed of the propeller is proportional to the wind speed and can be measured by methods similarly implemented with the cup anemometer. Both the cup and propeller anemometer are difficult to calibrate because factors such as friction, vary from instrument to instrument. Therefore, these mechanical anemometers must be calibrated in a controlled environment, such as a wind tunnel, wherein the air speed is measured by a pressure anemometer.

A pitot-static pressure tube anemometer is another mechanically simple instrument having no moving parts. The pitot-static anemometer is used as an air speed indicator in wind tunnels and aircraft. A probe having two separate tubes is aligned into the wind. The pitot tube is open at one end to allow air to blow in and cause a pressure build up. The pressure is the sum of the static air pressure and the dynamic air pressure produced by the flow of air into the tube. The other tube is a static tube that is closed and rounded at its upstream end with a series of holes around the tube a distance downstream. Near the rounded front of the static tube, the airstream is effectively undisturbed so the tube substantially contains only air at the static pressure. Between the downstream ends of the tubes is a differential pressure gauge. The dynamic pressure of the pitot tube is determined by measuring the pressure difference between the two tubes. The pitot-static anemometer can cause disturbances in the airstream and is generally not appropriate for measuring air velocity in confined spaces where such disturbances can be significant.

Measuring fluid flow volume is also desirable for much smaller applications than those generally discussed above. For instance, dispensing medicine to an individual requires control and monitoring of the prescribed dosage. Included among some of the more common dispensing techniques are oral, intravenous, respiratory and topical applications. Obviously, the accuracy of the dosage amount dispensed is dependent upon the type of technique and device utilized.

The amount of dosage for oral or intravenous ingestion is more easily controlled as compared to respiratory ingestion. The chemical dosage dispensed orally or intravenously is accurately controlled through tablet size and fluid flow volume measurement. Topical dispensation of a chemical can require the user to wear an unattractive patch for an extended period of time. The patch can also be uncomfortable to wear. In addition, the patch is susceptible to accidental removal during participation in physical activities such as exercising, swimming and running.

Inhalation into one's respiratory system is another technique used for administering a prescribed chemical to an individual. The chemical amount ingested through inhalation is conditioned upon a user's lung capacity and amount of inhalation. The rate and amount of inhale of an individual will also affect the amount of chemical actually ingested by the user. Individuals with larger lung capacity are able to ingest the prescribed chemical dosage in one or two inhales, while individuals with smaller lung capacity may not.

Some of the most common chemical inhalers merely dispense a predetermined amount of chemical into a receptacle for ingestion by the user. The user can fail to ingest the prescribed amount of chemical by improperly using the inhaler or insufficiently inhaling the dispensed amount. Any unused portion of the dispensed chemical can adversely affect the individual because the prescribed dosage will not be ingested. In addition, inefficient dispensing of the chemical will increase the cost of care for individuals using this technique.

The present invention is directed to solving these and other related problems.

### SUMMARY OF THE INVENTION

The apparatus of the present invention is directed to monitoring a pressure drop between adjacent regions. Specifically, the apparatus comprises a housing having a first and a second region, each region having an atmospheric pressure. A pressure sensor is operably attached between the first and second regions. The pressure sensor is responsive to a difference in atmospheric pressure between the first and second regions. A capacitance proportional to the pressure difference between the first and second regions is operably responsive to the pressure sensor. The capacitance is utilized by a measurement device to detect the pressure difference between the two regions.

A further aspect of the present invention is directed to the pressure sensor being a motor assembly. The motor assembly includes a diaphragm and a backplate. The diaphragm is operably attached to the backplate and spaced a distance from the backplate. The backplate is also connected to the capacitance measurement device. A fluid is capable of moving between the diaphragm and the backplate.

According to another aspect of the present invention, an apparatus for dispensing a chemical dosage comprises a housing having a first region and a second region, each region having an atmospheric pressure. A motor assembly is operably attached to the housing adjoining the two regions. The motor assembly is responsive to the atmospheric pressure of the first and second regions. The motor assembly comprises a diaphragm and a backplate. The backplate is operably connected to the diaphragm and is spaced a distance therefrom, wherein a fluid, preferably air, is capable of moving between the diaphragm and the backplate. A capacitance is responsive to the motor assembly and utilized by a measurement device to determine the pressure difference between the two regions. The capacitance is responsive to the spacial relationship between the diaphragm and the backplate. The capacitance is utilized by the measurement device for determining the chemical dosage to be dispensed. The chemical dosage is dispensed into the housing to be inhaled by the user. Using theoretical and empirical data, various chemical dosages can be correlated to the fluid flow volume and/or pressure difference detected within the housing. The chemical dosage dispensed within the housing is responsive to an individual's inhalation. The apparatus can also display the dosage amount dispensed to the individual.

Yet another aspect of the present invention is directed to a method of dispensing a chemical dosage to an individual. An inhaler is provided to the individual for inhaling the chemical dosage. The inhaler comprises a sensor for monitoring the inhalation of the individual. The inhalation is detected and the appropriate chemical dosage is dispensed to the individual in response to the inhalation. The chemical dosage is dispensed to the user by injecting the chemical dosage into the inhaler. Because of the low costs associated with the simple motor assembly discussed above, the apparatus of the present invention can be considered a disposable device.

One object of the present invention is to provide a motor assembly for detecting a difference in atmospheric pressure between two regions.

Another object of the present invention is to facilitate a simple, accurate, inexpensive, convenient, and disposable solution to dispensing a chemical dosage to an individual.

Other features and advantages of the invention will be apparent from the specification taken in conjunction with the following drawings.

### BRIEF DESCRIPTION OF THE DRAWINGS

FIG. 1A is a cross-sectional view of a venturi flowmeter;

FIG. 1B is a cross-sectional view of a flow nozzle flowmeter;

FIG. 1C is a cross-sectional view of an orifice flowmeter;

FIG. 2 is a cross-sectional view of one embodiment of the present invention;

FIG. 3 is a cross-sectional view of one embodiment of a motor assembly of the present invention;

FIG. 4 is a top-view of the motor assembly of FIG. 3;

FIG. 5 is a cross-sectional view of one embodiment of the motor assembly of the present invention; and,

FIG. 6 is a preferred embodiment of the motor assembly of the present invention.

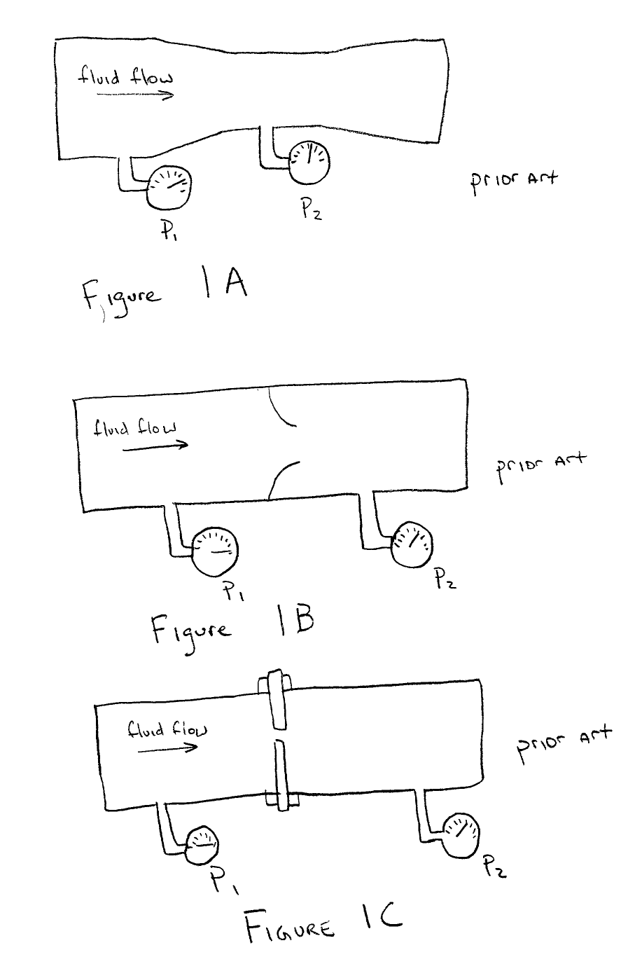

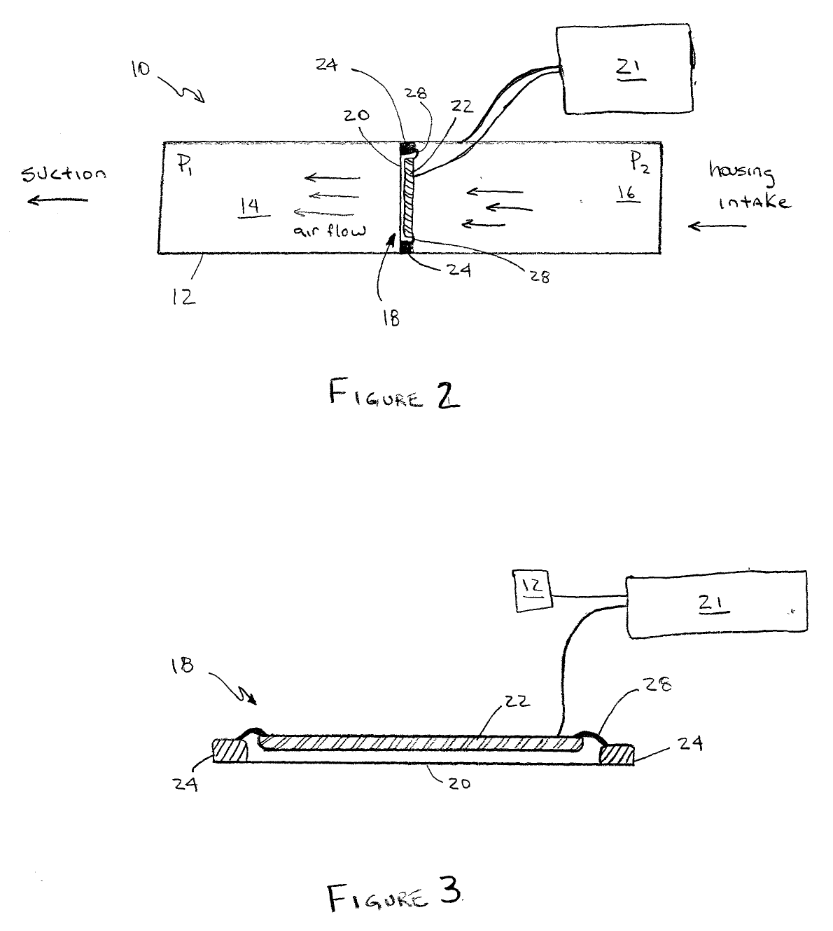

### DETAILED DESCRIPTION OF THE PREFERRED EMBODIMENT

While this invention is susceptible of embodiments in many different forms, there is shown in the drawings and will herein be described in detail, a preferred embodiment of the invention with the understanding that the present disclosure is to be considered as an exemplification of the principles of the invention and is not intended to limit the broad aspect of the invention to the embodiments illustrated.

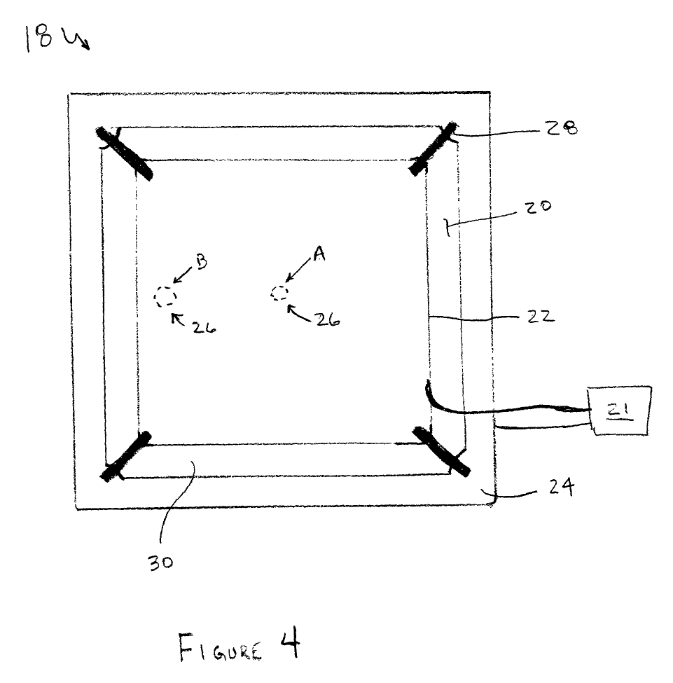

Referring now in detail to the FIGURES, and initially to FIG. 2, there is shown an apparatus 10 comprising a housing 12 having a first 14 and a second region 16, each region having an atmospheric pressure. A sensor 18 operably attached to the housing 12 adjoins the first 14 and second 16 regions and is responsive to the atmospheric pressure of the first and second regions. A capacitance is responsive to the sensor 18 wherein fluid movement affecting the pressure sensor 18 affects the capacitance. A measurement device 21 is operably connected to the sensor 18 and detects changes in the capacitance in response to the atmospheric pressure of the two regions 14, 16 affecting the sensor 18.

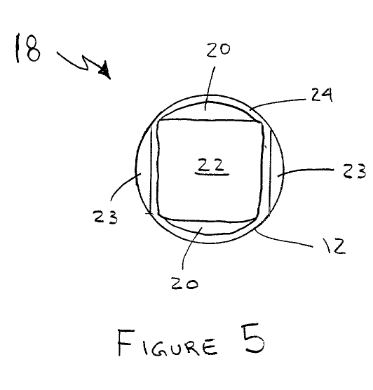

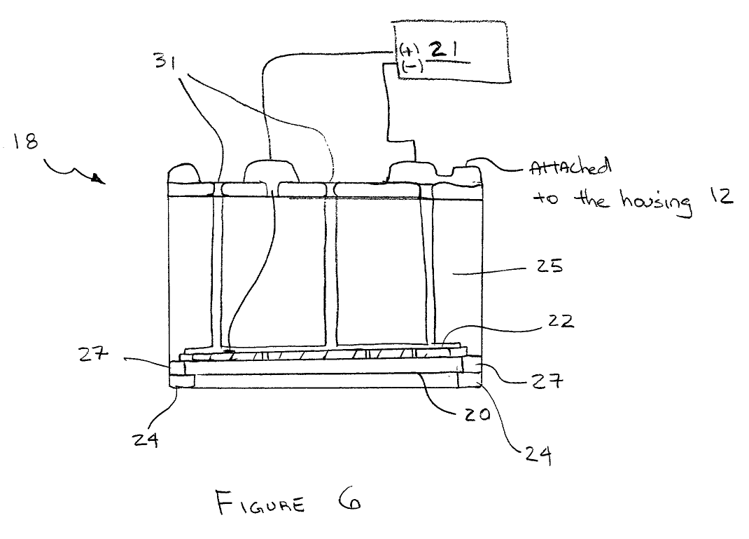

In one embodiment of the present invention, the pressure sensor 18 is a motor assembly similar to that shown in FIGS. 3, 5 and 6. The motor assembly 18 comprises a diaphragm 20 and a backplate 22. The backplate 22 is spaced a predetermined distance from the diaphragm 20 wherein air is capable of moving between the diaphragm and the backplate. A capacitance exists between the diaphragm 20 and the backplate 22. The motor assembly 18 is operably attached within the housing 12 and substantially perpendicular to the direction of fluid flow. The housing 12 and motor assembly 18 can be designed to partially inhibit the flow of air through the apparatus 10. Preferably, a vent 23 between the motor 18 and housing 12 allows the fluid to flow around the motor assembly 18. FIG. 5.

The motor assembly 18 is responsive to fluid movement within the housing 12 such that the fluid influences the spatial relationship between the diaphragm 20 and the backplate 22. Movement of the diaphragm 20 with respect to the backplate 22 alters the spatial relationship between the diaphragm and the backplate of the motor assembly 18. The changing spatial relationship affects the capacitance. The capacitance is monitored by a capacitance measurement device 21 wherein changes to the spatial relationship of the backplate 22 and diaphragm 20 will be detected. The diaphragm 20 and backplate 22 can be various shapes, such as square, rectangular, circular, triangular, elliptical, oval or a combination thereof.

Preferably, the motor assembly 18 includes a backplate 22 mounted to a support member, or a frame 25. A diaphragm 20 is operably connected to a ring 24. The ring conductively connects the diaphragm 20 to the housing 12. The backplate 22 is spaced a distance from the diaphragm 20 to provide a gap between the backplate and the diaphragm 20. The spacing between diaphragm 20 and the backplate 22 can be accomplished using a spacer 27, ring 24, frame 25 and a combination thereof. FIG. 6. A conduit 31 within the frame 25 allows the fluid flow pressure of the region 14, 16 to be present near the backplate 22. The distance of spacing between the backplate 22 and the diaphragm 20 is determined in cooperation with the size and shape of the housing 12, the motor assembly 18 and the specified application. In such an embodiment, the backplate 22 does not contact the diaphragm 20. The diaphragm 20 is free to move in response to the difference in air pressure between the first and second regions 14, 16 caused by fluid flow within the housing 12.

Another embodiment contemplated by the present invention is directed to a chemical delivery system for an individual. FIG. 2. The delivery system, preferably an inhaler 10, detects the amount of suction applied to one end of a “tube-like” housing 12. A motor assembly 18 defines a first region 14 and a second region 16 within the housing 12. The motor assembly 18 includes a diaphragm 20 operably connected to a backplate 22. The backplate 22 is spaced a predetermined distance from the diaphragm 20. Sensing circuitry, i.e., a capacitance measurement device 21, is operably connected to the motor assembly 18. A capacitance is responsive to the spatial relationship of the diaphragm 20 and the backplate 22. The backplate 22 is connected to the positive terminal (+) of the measurement device 21 and the diaphragm 20 is conductively connected to the housing 12 and the negative terminal (−) of the measurement device 21. The diaphragm 20 deflects in response to the difference in atmospheric pressure between the first 14 and second 16 regions, thus affecting the spatial relationship of the diaphragm 20 and backplate 22 and further affecting the capacitance. The capacitance measurement device 21 senses the capacitance and utilizes theoretical and empirical data to determine the chemical amount to be dispensed within the inhaler 12 for ingestion by the user. Preferably, the dispensed chemical is atomized and injected into the housing 12 for ingestion by the user.

The inhaler 10 of the present invention provides the user with the ability to control and monitor the chemical amount ingested over a period of time. The inhaler 10 facilitates an accurate and inexpensive dispensing device that can be disposed when empty. The volume of fluid flow within the housing 12 is determined by utilizing the capacitance responsive to the motor assembly 18. In response to the level of suction created by an individual, the level of capacitance is detected and a prescribed chemical dosage is determined. The dosage is dispensed within the housing 12 to be inhaled by the user.

As noted above, the apparatus 10 can be altered for various fluid flow volumes within the housing 12. For instance, the size of the motor assembly 18 can be varied, as well as the volume and shape of each atmospheric region 14, 16 of the housing 12. Thus, several inhaling sensations can be offered to the user varying from experiencing very little resistance during inhalation, i.e., sucking air through a straw; to experiencing an increased resistance, i.e., sucking a liquid through a straw.

An additional embodiment of the present invention is illustrated in FIG. 3. An apparatus 10 comprises a transducer having a motor assembly 18 attached to a housing 12. The motor assembly 18 divides the interior of the housing 12 into a first atmospheric 14 and a second atmospheric region 16. The motor assembly 18 comprises a diaphragm 20, a support member 24, and a backplate 22. Additionally, the transducer can include a support plate for supporting an amplifier that is operably connected to the backplate 22.

The support member 24, also referred to as a diaphragm ring, has a first side, a second side, and an aperture 30 extending from the first side through to the second side. As shown in FIG. 2, the support member 24 is conductively attached in the housing 12 and secured with a conductive adhesive.

The motor assembly 18 includes a diaphragm 20 operably connected to the support member 24 at a periphery portion which is adhered to the second side of the support member adjacent the aperture 30 in the support member. FIGS. 3 and 4. As such, the central portion of the diaphragm 20 substantially covers the aperture 30 in the support member 24 and is capable of vibrating thereabout. Preferably, nothing contacts the central portion of the diaphragm 20 adjacent the aperture 30 in the support member 24. The diaphragm 20 is operably connected to the housing 12. The backplate 22 is suspended from the support member 24. A non-conductive adhesive 28 or some other connection means connects the backplate 22 to the support member 24 in a spaced relation. An adhesive material is applied to the periphery of the backplate 22 and support member 24 respectively, in a bridge-like manner to hold the backplate 22 in place.

Yet another embodiment of the motor assembly 18 incorporates a pierce hole 26 that extends through the central portion of the diaphragm 20 adjacent the aperture 30 in the support member 24. FIG. 4. The pierce hole 26 provides barometric relief. One of two locations is utilized for the pierce hole 26, location “A” which is generally centrally located on the diaphragm 20, and location “B” which is located on the centerline of the diaphragm 20, adjacent the support member 24.

While the specific embodiment has been illustrated and described, numerous modifications come to mind without significantly departing from the spirit of the invention, and the scope of protection is only limited by the scope of the accompanying Claims.

I claim:

1. A pressure sensor for measuring pressure differences between a first and second region, the pressure sensor being operably connected to a capacitive measurement device, the pressure sensor comprising:
a motor operably attached between the first and second regions, the motor being responsive to a difference in atmospheric pressure between the first and second regions; and,
a capacitance operably responsive to the motor wherein the capacitance is detected by the measurement device for determining the pressure difference between the two regions.

2. The pressure sensor of claim 1 wherein the motor comprises:
a diaphragm; and,
a backplate operably attached to the diaphragm and spaced a distance from the diaphragm, wherein a fluid is capable of moving between the diaphragm and the backplate, the motor being operably connected to the measurement device.

3. The pressure sensor of claim 2 wherein the motor comprises a support member, the support member being operably attached to a housing, the housing substantially enclosing the first and second regions.

4. An inhaler for dispensing a chemical dosage, the inhaler being operably connected to a capacitance measurement device, the inhaler comprising:
a housing having a first region and a second region, each region having an atmospheric pressure;
a motor assembly operably attached within the housing and adjoining the first region and the second region, the motor assembly being responsive to the atmospheric pressure of each region; and,
a capacitance operably responsive to the motor assembly wherein the capacitance measurement device detects a pressure difference between the first and second regions for determining the chemical dosage to be dispensed.

5. The inhaler of claim 4 wherein the motor assembly comprises:
a diaphragm operably connected to the housing; and,
a backplate operably connected to the capacitance measurement device, the backplate being spaced a distance from the diaphragm wherein a fluid is capable of moving between the diaphragm and the backplate.

6. The inhaler of claim 5 wherein the motor assembly comprises a support member operably attached to the housing, the support member operably adjoining the diaphragm and the backplate.

7. An inhaler for dispensing an a chemical dosage to a user, the inhaler being operably connected to a capacitance measurement device, the inhaler comprising:
a housing;
a motor assembly operably attached to the housing, the motor assembly defining a first region and a second region within the housing, each region having an atmospheric pressure; and,
a capacitance operably responsive to the motor assembly wherein a difference in atmospheric pressure between the first region and the second region is sensed by the motor assembly, the capacitance being detected by the capacitance measurement device wherein the amount of the chemical to be dispensed to the user is determined in response to the capacitance detected.

8. The inhaler of claim 7 wherein the motor assembly comprises:
a diaphragm; and,
a backplate operably attached to the diaphragm and spaced a distance therefrom, the motor assembly being operably connected to the measurement device, wherein the capacitance is responsive to the distance between the diaphragm and the backplate.

9. The inhaler of claim 8 wherein the motor assembly comprises a support member operably attached to the housing, the support member operably adjoining the diaphragm and the backplate.

10. A method of dispensing a chemical dosage to an individual comprising the steps of:
providing an inhaler to the individual, the inhaler having a first and second region, each region having a pressure;
detecting a difference in pressure between the first and second region; and,
dispensing the chemical dosage to the individual in response to the detected pressure difference.

11. The method of claim 10 wherein the step of dispensing the chemical dosage to the individual comprises injecting the chemical dosage into the inhaler.

12. The method of claim 10 wherein the inhaler comprises:
a housing;
a motor assembly operably attached within the housing, the motor assembly defining a first region and a second region, each region having an atmospheric pressure; and,
a capacitance operably responsive to the motor assembly wherein a difference in atmospheric pressure between the first region and the second region is sensed by the motor assembly, the capacitance being detected by a capacitance measurement device wherein the amount of the chemical to be dispensed to the user is determined in response to the capacitance detected.

13. The method of claim 12 wherein the motor assembly comprises:
a diaphragm; and,
a backplate operably attached to the diaphragm and spaced a distance therefrom, the motor assembly being operably connected to the measurement device, wherein the capacitance is responsive to the distance between the diaphragm and the backplate.
